(19) United States
(12) Patent Application Publication
(10) Pub. No.: US 2006/0287234 A1
(43) Pub. Date: Dec. 21, 2006

(54) Wound healing

(75) Inventors: Timothy Breen, Half Moon Bay, CA (US); Stuart Bunting, Half Moon Bay, CA (US); Charles Semba, Palo Alto, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/455,017

(22) Filed: Jun. 16, 2006

Related U.S. Application Data
(60) Provisional application No. 60/794,008, filed on Apr. 21, 2006.
(60) Provisional application No. 60/691,909, filed on Jun. 17, 2005.

(51) Int. Cl. A61K 38/17

(52) U.S. Cl. 514/12

(57) ABSTRACT

Methods for accelerating and/or improving wound healing in a subject by administering vascular endothelial growth factor (VEGF) are provided.

### RELATED APPLICATION

This application claims priority to and the benefit of U.S. Provisional Application Ser. No. 60/691,909, filed Jun. 17, 2005, and U.S. Provisional Application Ser. No. 60/794,008, filed Apr. 21, 2006, the specifications of which are incorporated herein in their entirety.

### FIELD OF THE INVENTION

The invention relates to methods of accelerating or improving wound healing by administering vascular endothelial growth factor (VEGF).

### BACKGROUND

Wound healing is a complex process, involving an inflammation phase, a granulation tissue formation phase, and a tissue remodeling phase. Singer and Clark, Cutaneous Wound Healing, N. Engl. J. Med. 341:738-46 (1999). These events are triggered by cytokines and growth factors that are released at the site of injury. Many factors can complicate or interfere with normal adequate wound healing. For example, such factors include age, infection, poor nutrition, immunosuppression, medications, radiation, diabetes, peripheral vascular disease, systemic illness, smoking, stress, etc.

For patients with diabetes, which is a chronic, debilitating disease that will affect approximately 20 million people in the United States in 2005, development of a diabetic foot ulcer (also referred to as a wound) is a common complication. A chronic ulcer is defined as a wound that does not proceed through an orderly and timely repair process to produce anatomic and functional integrity (see, e.g., Lazarus et al., Definitions and guidelines for assessment of wounds and evaluation of healing, Arch. Dermatol. 130:489-93 (1994)). By its nature, the diabetic foot ulcer is a chronic wound (American Diabetes Association, Consensus development conference on diabetic foot wound care, Diabetes Care, 22(8):1354-60 (1999)). Because the skin serves as the primary barrier again the environment, an open refractory wound can be catastrophic; a major disability (including limb loss) and even death can result. Foot ulceration is the precursor to about 85% of lower extremity amputations in persons with diabetes. See, e.g., Apelqvist, et al., What is the most effective way to reduce incidence of amputation in the diabetic foot? Diabetes Metab Res. Rev., 16(1 Suppl.): S75-S83 (2000).

It has been reported that there are over thirty-five million cutaneous wounds requiring intervention annually in the US. See, e.g., Tonnesen et al., Angiogenesis in Wound Healing JID Symposium Proceedings 5(1):40-46 (2000). Current wound care therapies have not been very successful due to their disappointing efficacy and to their cost. Thus, there is a need to enhance and optimize wound healing therapies for subjects. The present invention addresses these and other needs, as will be apparent upon review of the following disclosure.

### SUMMARY

Methods for accelerating the healing of wounds, e.g., acute (e.g., burn, surgical wound, etc.) or chronic (e.g., diabetic ulcer, pressure ulcer, a decubitus ulcer, a venous ulcer, etc.), or normal, are provided. Methods for improving wound healing along and reducing the amount of recurrences of ulcers with the administration of vascular endothelial growth factor (VEGF) are also provided. Methods include, e.g., a method of accelerating wound healing in a subject, where a method comprises administering an effective amount of VEGF to a wound, where the administration of the effective amount of VEGF accelerates wound healing greater than 50%, or equal to or greater than 60%, equal to or greater than 70%, equal to or greater than 74%, equal to or greater than 75%, equal to or greater than 80%, equal to or greater than 85%, equal to or greater than 90%, equal to or greater than 95%, equal to or greater than 100%, equal to or greater than 110% or more, when compared to a control. A control includes, but is not limited to, e.g., a subject who is not administered treatment, or a subject who is administered sub-therapeutic amount of VEGF, or a subject who is administered another wound treatment, or a subject who is administered a placebo, either with or without Good Wound Care (GWC), or a subject who is administered GWC alone. GWC can include, but is not limited to, e.g., debridement, cleaning/dressings, pressure relief, infection control, and/or combinations thereof. In one embodiment, a method of accelerating wound healing in a human subject includes administering an effective amount of VEGF to a wound, wherein the administration of the effective amount of VEGF accelerates wound healing greater than 60% when compared a control and wherein the wound is present on the subject for about 4 weeks or more before administering the effective amount of VEGF. In one embodiment, a method of accelerating wound healing in a human subject includes administering an effective amount of rhVEGF165 to a diabetic wound, where the administration of the effective amount of rhVEGF165 accelerates wound healing greater than 60% when compared a control. In certain embodiments, a VEGFR agonist can be used in place of or with VEGF in the methods.

Assessment of wound healing can be determined, e.g., by the % reduction in the wound area, or complete wound closure. The wound area can be determined by quantitative analysis, e.g., area measurements of the wound, planimetric tracings of the wound, etc. Complete wound closure can be determined by, e.g., skin closure without drainage or dressing requirements. Photographs of the wound, physical examinations of the wound, etc. can also be used to assess wound healing. Acceleration of wound healing can be expressed in terms of % acceleration or expressed in terms of a Hazard ratio as a time to healing (e.g., VEGF verses a control, e.g., a placebo), etc. In certain embodiments of the invention, the Hazard ratio (HR) is greater than or equal to 1.75, or greater than or equal to 1.8, or greater than or equal to 1.85, or greater than or equal to 1.87, or greater than or equal to 1.9, or greater than or equal to 1.95, or greater than or equal to 1.98, or greater than or equal to 2.0, or greater than or equal to 2.1 or more.

In one embodiment, the wound further comprises an infection. In another embodiment, the wound is an ischemic wound. In one embodiment, the wound area before treatment is about 0.4 cm² or more, or about 1.0 cm² or more, or between about 1.0 cm² and about 10.0 cm², or between about 1.0 cm² and about 6.5 cm², or between about 1.0 cm² and about 5.0 cm². In a further embodiment, the wound area is determined before treatment with VEGF but after debridement. In one embodiment, the wound is present on the subject for about 4 weeks or more, or about 6 weeks or more, before administering the VEGF. In certain aspects of the invention, the subject is or has undergone a treatment, where the treatment delays or provides ineffective wound healing. In another embodiment, the subject has a secondary condition, wherein the secondary condition delays or provides ineffective wound healing. In a further embodiment, the secondary condition is diabetes.

In one embodiment, the VEGF administered is VEGF₁₆₅ (e.g., recombinant human VEGF (e.g., human VEGF₁₆₅)). In one embodiment, the VEGF is administered topically. In certain embodiments, VEGF is administered in combination with other factors that accelerate wound healing (e.g., angiogenesis factor or agent, wound healing agent or procedure, growth factor, etc.). The VEGF can be formulated in, e.g., a slow-release formulation, a gel formulation, a bandage or dressing, etc. In certain embodiments, the subject is a human. In one embodiment, the effective amount of VEGF administered is about 20 μg/cm² to about 250 μg/cm². In certain embodiments, the effective amount administered is about 24 μg/cm², or 24 μg/cm². In certain embodiments, the effective amount administered is about 72 μg/cm², or 72 μg/cm². In certain embodiments, the effective amount administered is about 216 μg/cm², or 216 μg/cm². In one embodiment, the effective amount of VEGF administered is 20 μg/cm² to 250 μg/cm². In certain embodiments, the effective amount administered is about 24 μg/cm² to about 216 μg/cm², or 24 μg/cm² to 216 μg/cm² In certain embodiments, the effective amount administered is about 24 μg/cm² to about 72 μg/cm², or 24 μg/cm² to 72 μg/cm². In certain embodiments, the effective amount administered is about 72 μg/cm² to about 216 μg/cm², or 72 μg/cm² to 216 μg/cm². In certain embodiments, the effective amount administered is about 216 μg/cm² to about 250 μg/cm², or 216 μg/cm² to 250 μg/cm².

The administration of the effective amount of VEGF can be daily or optionally a few times a week, e.g., at least twice a week, or at least three times a week, or at least four times a week, or at least five times a week, or at least six times a week. In one embodiment, VEGF is administered for at least six weeks, or greater than six weeks, or at least about twelve weeks, or until complete wound closure (e.g., which can be determined by skin closure without drainage or dressing requirements). In one embodiment, VEGF is administered for less than 20 weeks for one treatment course.

Methods of the invention also include a method of improving wound healing in a population of subjects. For example, a method comprises administering an effective amount of VEGF to a wound of a subject of the population, where the administration of the effective amount of VEGF results in greater than 10% (or greater than 12%, or 14%, or 15%, or 17%, or 20%, or 25%, or 30%, or 33%, or 35%, or 40%, or 45%, or 50% or more) improvement in wound healing in the population compared to a control population. For example, a control population includes, but is not limited to, e.g., subjects who are not administered treatment, or subjects who are administered sub-therapeutic amount of VEGF, or subjects who are administered another wound treatment, or subjects who are administered a placebo, either with or without Good Wound Care (GWC), or subjects who are administered GWC alone. In one embodiment, improved wound healing is assessed by complete wound healing. In certain embodiments of the invention, the population includes subjects with impaired wound healing. In one embodiment, the population is diabetic patients with chronic wounds, e.g., for about 4 weeks or more before treatment.

Methods for reducing the recurrence of ulcers are also provided by the invention. For example, a method comprises administering an effective amount of VEGF to an ulcer, where the incidence of ulcer formation is reduced with VEGF administration compared to a control. For example, a control includes, but is not limited to, e.g., a subject who is not administered treatment, or a subject who is administered sub-therapeutic amount of VEGF, or a subject who is administered another wound treatment, or a subject who is administered a placebo, either with or without Good Wound Care (GWC), or a subject who is administered GWC alone.

### BRIEF DESCRIPTION OF THE FIGURES

FIG. 1 illustrates a study design, e.g., VGF 2763g, for administering rhVEGF for the treatment of diabetic wounds.

FIG. 2 illustrates dose-response curve of the addition of rhVEGF in a rabbit ischemic ear wound model at day 14.

FIG. 3 illustrates a does-response curve of the addition of rhVEGF in a diabetic mouse model at day 8.

### DETAILED DESCRIPTION

Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “VEGF” (also referred to as VEGF-A) as used herein refers to vascular endothelial cell growth factor protein. The term “human VEGF” (also referred to as human VEGF-A) as used herein refers to the 165-amino acid human vascular endothelial cell growth factor, and related 121-, 145-, 183-189-, and 206-, (and other isoforms) amino acid vascular endothelial cell growth factors, as described by Leung et al., Science 246:1306 (1989), and Houck et al., Mol. Endocrin. 5:1806 (1991) together with the naturally occurring allelic and processed forms of those growth factors.

A “native sequence” polypeptide comprises a polypeptide having the same amino acid sequence as a polypeptide derived from nature. Thus, a native sequence polypeptide can have the amino acid sequence of naturally occurring polypeptide from any mammal. Such native sequence polypeptide can be isolated from nature or can be produced by recombinant or synthetic means. The term “native sequence” polypeptide specifically encompasses naturally occurring truncated or secreted forms of the polypeptide (e.g., an extracellular domain sequence), naturally occurring variant forms (e.g., alternatively spliced forms) and naturally occurring allelic variants of the polypeptide.

A polypeptide “variant” means a biologically active polypeptide having at least about 80% amino acid sequence identity with the corresponding native sequence polypeptide, or fragment thereof. Such variants include, for instance, polypeptides wherein one or more amino acid residues are added, or deleted, at the N- and/or C-terminus of the polypeptide. Ordinarily, a variant will have at least about 80% amino acid sequence identity, or at least about 90% amino acid sequence identity, or at least about 95% or more amino acid sequence identity with the native sequence polypeptide, or fragment thereof. Analogues or variants are defined as molecules in which the amino acid sequence, glycosylation, or other feature of native VEGF has been modified covalently or noncovalently.

The term “VEGF variant” as used herein refers to a variant as described above and/or an VEGF which includes one or more amino acid mutations in the native VEGF sequence. Optionally, the one or more amino acid mutations include amino acid substitution(s). VEGF and variants thereof for use in the invention can be prepared by a variety of methods well known in the art. Amino acid sequence variants of VEGF can be prepared by mutations in the VEGF DNA. Such variants include, for example, deletions from, insertions into or substitutions of residues within the amino acid sequence of VEGF, e.g., a human amino acid sequence encoded by the nucleic acid shown in 5,332,671; 5,194,596; or 5,240,848. Any combination of deletion, insertion, and substitution may be made to arrive at the final construct having the desired activity, e.g., VEGF activity, e.g., accelerating wound healing. The mutations that will be made in the DNA encoding the variant must not place the sequence out of reading frame and preferably will not create complementary regions that could produce secondary mRNA structure. EP 75,444A. VEGF variants can be assessed for VEGF activity, e.g., by a cell proliferation assay. For example, a cell proliferation assay includes increasing the extent of growth and/or reproduction of the cell relative to an untreated cell or a reduced treated cell either in vitro or in vivo. An increase in cell proliferation in cell culture can be detected by counting the number of cells before and after exposure to a molecule of interest. The extent of proliferation can be quantified via microscopic examination of the degree of confluence. Cell proliferation can also be quantified using the thymidine incorporation assay.

The VEGF variants optionally are prepared by site-directed mutagenesis of nucleotides in the DNA encoding the native VEGF or phage display techniques, thereby producing DNA encoding the variant, and thereafter expressing the DNA in recombinant cell culture.

While the site for introducing an amino acid sequence variation is predetermined, the mutation per se need not be predetermined. For example, to optimize the performance of a mutation at a given site, random mutagenesis may be conducted at the target codon or region and the expressed VEGF variants screened for the optimal combination of desired activity. Techniques for making substitution mutations at predetermined sites in DNA having a known sequence are well-known, such as, for example, site-specific mutagenesis. Preparation of the VEGF variants described herein can be achieved by phage display techniques, such as those described in the PCT publication WO 00/63380.

After such a clone is selected, the mutated protein region may be removed and placed in an appropriate vector for protein production, generally an expression vector of the type that may be employed for transformation of an appropriate host.

Amino acid sequence deletions generally range from about 1 to 30 residues, optionally 1 to 10 residues, optionally 1 to 5 or less, and typically are contiguous.

Amino acid sequence insertions include amino- and/or carboxyl-terminal fusions of from one residue to polypeptides of essentially unrestricted length as well as intrasequence insertions of single or multiple amino acid residues. Intrasequence insertions (i.e., insertions within the native VEGF sequence) may range generally from about 1 to 10 residues, optionally 1 to 5, or optionally 1 to 3. An example of a terminal insertion includes a fusion of a signal sequence, whether heterologous or homologous to the host cell, to the N-terminus to facilitate the secretion from recombinant hosts.

Additional VEGF variants are those in which at least one amino acid residue in the native VEGF has been removed and a different residue inserted in its place. Such substitutions may be made in accordance with those shown in Table 1. VEGF variants can also comprise unnatural amino acids as described herein.

Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Trp (W), Met (M)

(2) uncharged polar: Gly (G), Ser (S), Thr (T), Cys (C), Tyr (Y), Asn (N), Gln (Q)

(3) acidic: Asp (D), Glu (E)

(4) basic: Lys (K), Arg (R), His(H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

“Naturally occurring amino acid residues” (i.e. amino acid residues encoded by the genetic code) may be selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val). A “non-naturally occurring amino acid residue” refers to a residue, other than those naturally occurring amino acid residues listed above, which is able to covalently bind adjacent amino acid residues(s) in a polypeptide chain. Examples of non-naturally occurring amino acid residues include, e.g., norleucine, ornithine, norvaline, homoserine and other amino acid residue analogues such as those described in Ellman et al. Meth. Enzym. 202:301-336 (1991) & US Patent application publications 20030108885 and 20030082575. Briefly, these procedures involve activating a suppressor tRNA with a non-naturally occurring amino acid residue followed by in vitro or in vivo transcription and translation of the RNA. See, e.g., US Patent application publications 20030108885 and 20030082575; Noren et al. Science 244:182 (1989); and, Ellman et al., supra.

“Percent (%) amino acid sequence identity” herein is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2. The ALIGN-2 sequence comparison computer program was authored by Genentech, Inc. has been filed with user documentation in the U.S. Copyright Office, Washington D.C., 20559, where it is registered under U.S. Copyright Registration No. TXU510087, and is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

The term “VEGF receptor” or “VEGFR” as used herein refers to a cellular receptor for VEGF, ordinarily a cell-surface receptor found on vascular endothelial cells, as well as variants thereof which retain the ability to bind VEGF.

The term “VEGFR agonist” refers to a molecule that can activate a VEGF receptor or increase its expression. VEGFR agonists include, but are not limited to, e.g., ligand agonists of a VEGFR, VEGF variants, antibodies and active fragments. VEGF is a VEGFR agonist, but herein it is separately listed and referred to. The term “Anti-VEGFR antibody” is an antibody that binds to VEGFR with sufficient affinity and specificity. In one embodiment, the anti-VEGFR agonist antibody of the invention can be used as a therapeutic agent in treating wounds. In another embodiment, a VEGF variant can be used as a therapeutic agent in treating wounds.

The term “antibody” is used in the broadest sense and includes monoclonal antibodies (including full length or intact monoclonal antibodies), polyclonal antibodies, multivalent antibodies, multispecific antibodies (e.g., bispecific antibodies), and antibody fragments so long as they exhibit the desired biological activity.

Unless indicated otherwise, the expression “multivalent antibody” is used to denote an antibody comprising three or more antigen binding sites. The multivalent antibody is typically engineered to have the three or more antigen binding sites and is generally not a native sequence IgM or IgA antibody.

“Antibody fragments” comprise only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)2 fragments, a bivalent fragment including two Fab′ fragments linked by a disulphide bridge at the hinge region; (ix) single chain antibody molecules (e.g. single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH—CH1-VH—CH1) which, together with complementary light chain polypeptides, form a pair of antigen binding regions (Zapata et al. Protein Eng. 8(10):1057 1062 (1995); and U.S. Pat. No. 5,641,870).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) or Marks et al., J. Mol. Biol. 222:581-597 (1991), for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 14:309-314 (1996): Sheets et al. PNAS (USA) 95:6157-6162 (1998)); Hoogenboom and Winter, J. Mol. Biol., 227:381 (1991); Marks et al., J. Mol. Biol., 222:581 (1991)). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

Diabetes is a chronic disorder affecting carbohydrate, fat and protein metabolism in animals. Diabetes is the leading cause of blindness, renal failure, and lower limb amputations in adults and is a major risk factor for cardiovascular disease and stroke.

Type I diabetes mellitus (or insulin-dependent diabetes mellitus (“IDDM”) or juvenile-onset diabetes) comprises approximately 10% of all diabetes cases. The disease is characterized by a progressive loss of insulin secretory function by beta cells of the pancreas. This characteristic is also shared by non-idiopathic, or “secondary”, diabetes having its origins in pancreatic disease. Type I diabetes mellitus is associated with the following clinical signs or symptoms, e.g., persistently elevated plasma glucose concentration or hyperglycemia; polyuria; polydipsia and/or hyperphagia; chronic microvascular complications such as retinopathy, nephropathy and neuropathy; and macrovascular complications such as hyperlipidemia and hypertension which can lead to blindness, end-stage renal disease, limb amputation and myocardial infarction.

Type II diabetes mellitus (non-insulin-dependent diabetes mellitus or NIDDM) is a metabolic disorder involving the dysregulation of glucose metabolism and impaired insulin sensitivity. Type II diabetes mellitus usually develops in adulthood and is associated with the body's inability to utilize or make sufficient insulin. In addition to the insulin resistance observed in the target tissues, patients suffering from type II diabetes mellitus have a relative insulin deficiency—that is, patients have lower than predicted insulin levels for a given plasma glucose concentration. Type II diabetes mellitus is characterized by the following clinical signs or symptoms, e.g., persistently elevated plasma glucose concentration or hyperglycemia; polyuria; polydipsia and/or hyperphagia; chronic microvascular complications such as retinopathy, nephropathy and neuropathy; and macrovascular complications such as hyperlipidemia and hypertension which can lead to blindness, end-stage renal disease, limb amputation and myocardial infarction.

“Subject” for purposes of the invention refers to any animal. Generally, the animal is a mammal. “Mammal” for purposes of invention refers to any animal classified as a mammal, including humans, domestic and farm animals, and zoo, sports, or pet animals, such as dogs, horses, cats, cows, sheep, pigs, etc. Typically, the mammal is a human.

The term “accelerating wound healing” or “acceleration of wound healing” refers to the increase in the rate of healing, e.g., a reduction in time until complete wound closure occurs or a reduction in time until a % reduction in wound area occurs.

The term “effective amount” or “therapeutically effective amount” refers to an amount of a drug effective to accelerate or improve wound healing in a subject or prevent recurrence of an ulcer in a subject. A therapeutic dose is a dose which exhibits a therapeutic effect on the subject and a sub-therapeutic dose is a dose which does not exhibit a therapeutic effect on the subject treated.

Administration “in combination with” one or more further therapeutic agents includes simultaneous (concurrent) and/or consecutive administration in any order.

“Treatment” refers to both therapeutic treatment and prophylactic or preventative measures. Those in need of treatment include those already with the disorder as well as those in which the disorder is to be prevented.

“Wound healing” refers a condition that would benefit from treatment with a molecule of the invention.

A “chronic wound” refers a wound that does not heal. See, e.g., Lazarus et al., Definitions and guidelines for assessment of wounds and evaluation of healing, Arch. Dermatol. 130:489-93 (1994). Chronic wounds include, but are not limited to, e.g., arterial ulcers, diabetic ulcers, pressure ulcers, venous ulcers, etc. An acute wound can develop into a chronic wound. Acute wounds include, but are not limited to, wounds caused by, e.g., thermal injury, trauma, surgery, excision of extensive skin cancer, deep fungal and bacterial infections, vasculitis, scleroderma, pemphigus, toxic epidermal necrolysis, etc. See, e.g., Buford, Wound Healing and Pressure Sores, HealingWell.com, published on: Oct. 24, 2001. A “normal wound” refers a wound that undergoes normal wound healing repair.

“Good Wound Care (GWC)”refers to the steps to take care of a wound. For example, good wound care practices include, but are not limited to, one or more of the following, debridement (e.g., surgical/sharp, mechanical, autolytic or chemical/enzymatic), cleaning (e.g., routine wound cleansing with, e.g., saline), dressings, pressure relief (e.g., off-loading pressure to the foot), maintenance of moist wound environment, and/or infection control (e.g., antibiotic ointment or pills). Other steps optionally include fitting subject with comfortable, cushioned footwear, nutritional support, maintaining blood glucose control, management of other risk factors (e.g., weight, smoking), etc. GWC can include one or more of the practices.

The expression “trauma affecting the vascular endothelium” refers to trauma, such as injuries, to the blood vessels or heart, including the vascular network of organs, to which an animal or human, preferably a mammal and most preferably a human, is subjected. Examples of such trauma include wounds, incisions, and ulcers, or lacerations of the blood vessels or heart. Trauma includes conditions caused by internal events as well as those that are imposed by an extrinsic agent that can be improved by promotion of vascular endothelial cell growth.

An “angiogenic factor or agent” is a growth factor which stimulates the development of blood vessels, e.g., promotes angiogenesis, endothelial cell growth, stability of blood vessels, and/or vasculogenesis, etc. For example, angiogenic factors, include, but are not limited to, e.g., VEGF and members of the VEGF family, PIGF, PDGF family, fibroblast growth factor family (FGFs), TIE ligands (Angiopoietins), ephrins, ANGPTL3, ANGPTL4, etc. It also includes factors, such as growth hormone, insulin-like growth factor-I (IGF-I), VIGF, epidermal growth factor (EGF), CTGF and members of its family, and TGF-α and TGF-β. See, e.g., Klagsbrun and D'Amore, Annu. Rev. Physiol., 53:217-39 (1991); Streit and Detmar, Oncogene, 22:3172-3179 (2003); Ferrara & Alitalo, Nature Medicine 5(12):1359-1364 (1999); Tonini et al., Oncogene, 22:6549-6556 (2003) (e.g., Table 1 listing angiogenic factors); and, Sato Int. J. Clin. Oncol., 8:200-206 (2003).

Wound Healing

The healing a wound is a complex process that involves three major phases: inflammation, granulation tissue, and tissue remodeling. At the site of the wound there is many processes occurring, e.g., migration/contraction, matrix metalloproteases (MMP) production, proliferation, and angiogenesis. There is contraction (downsizing of the wound), epithelialization (creation of new epithelial cells) and deposition of connective tissue in order to heal the wound. See Singer & Clark, Cutaneous Wound Healing N. Engl. J. Med., 341:738-46 (1999). One of the goals of wound therapy is to promote the granulation matrix, where an adequate blood supply is needed. However, risk factors, often associated with diseases states, (e.g., include, but are not limited to, age, infection, poor nutrition, immunosuppression, medications, radiation, diabetes, peripheral vascular disease, systemic illness, smoking, stress, etc.) create challenges for wound healing.

Examples of some of the risk factors for diabetic foot ulcers include peripheral neuropathy, which affects both motor and sensory functions of the foot, limited joint mobility, foot deformities, abnormal distribution of foot pressure, repetitive minor trauma, and impaired visual acuity. See, e.g., Boyko et al., A prospective study of risk factors for diabetic foot ulcer, The Seattle Diabetic Foot Study, Diabetes Care, 22:1036-42 (1999); and Apelqvist et al., International consensus and practical guidelines on the management and the prevention of the diabetic foot, International Working Group on the Diabetic Foot., Diabetes Metab Res. Rev 16(1 Suppl): S84-S92 (2000). Peripheral sensory neuropathy is a primary factor. Approximately 45%-60% of all diabetic ulcerations are neuropathic, while up to 45% have both neuropathic and ischemic components. With an insensate foot, the patient is unable to perceive repetitive injury to the foot caused by, e.g., poor-fitting footwear during ambulation and activities of daily living. Neuropathy, combined with altered biomechanics of walking, leads to repetitive blunt trauma and distribution of abnormally high stress loads to vulnerable portions of the foot, resulting in callus formation and cutaneous erosion. Once an ulcer is formed, it is often slow to heal, can continue to enlarge, provides an opportunity for local or systemic infection, and requires comprehensive medical and surgical care to promote healing.

There are also challenges to creating protein therapies to accelerate wound healing, e.g., accelerating healing of chronic, e.g., diabetic foot ulcers, wounds. The wound area is a hostile environment (proteolytic enzymes, naturally produced inhibitors of protein activity along with superimposed infection), where often in disease states (e.g., in diabetes) the host factors are altered (e.g., in diabetes there is suppressed VEGF expression, impaired VEGF response to hypoxia, altered cellular metabolism, suppressed immune/inflammatory response, etc.). For example, based upon in vitro studies, keratinocytes and fibroblasts from diabetic (db/db) mice exhibit selective impairment of cellular processes essential for normal tissue repair, and db/db fibroblasts show significantly decreased cellular migration and growth factor alterations. See, e.g., Frank et al., Regulation of vascular endothelial growth factor expression in cultured keratinocytes, Implications for normal and impaired wound healing, J. Biol. Chem., 270:12607-13 (1995); and, Lerman et al, Cellular dysfunction in the diabetic fibroblast: impairment in migration, vascular endothelial growth factor production, and response to hypoxia, Am. J. Pathol., 162:303-12 (2003).

Provided herein are methods for accelerating and/or improving healing of wounds, by administering effective amount of VEGF or a VEGF agonist. For example, a method comprises administering an effective amount of VEGF to a wound of a subject, where the administration of the effective amount of VEGF accelerates wound healing. Methods also include a method of improving wound healing in a population of subjects. For example, a method comprises administering an effective amount of VEGF to a wound of a subject of the population, wherein the administration of the effective amount of VEGF results in greater than 10% (or greater than 12%, or 14%, or 15%, or 17%, or 20%, or 25%, or 30%, or 33%, or 35%, or 40%, or 45%, or 50% or more) improvement in wound healing in the population compared to a control. Methods for reducing the recurrence of ulcers are also provided. For example, a method comprises administering an effective amount of VEGF to an ulcer, wherein the incidence of ulcer recurrence is reduced with VEGF administration compared to a control.

Methods are also applicable to subjects who are undergoing or have undergone a treatment, where the treatment delays or provides ineffective wound healing. Treatments can include, but are not limited to, medications, radiation, treatments that results in suppressed immune systems, etc. Optionally, a subject of the invention has a secondary condition, wherein the secondary conditions delays or provides ineffective wound healing. Secondary conditions, include, but are not limited to, e.g., diabetes, peripheral vascular disease, infection, autoimmune or collagen vascular disorders, disease states that result in suppressed immune systems, etc.

Acceleration of wound healing can be described by % acceleration of wound healing and/or a Hazard ratio. In certain embodiments, the administration of the effective amount of VEGF accelerates wound healing greater than 50%, or equal to or greater than 60%, equal to or greater than 70%, equal to or greater than 74%, equal to or greater than 75%, equal to or greater than 80%, equal to or greater than 85%, equal to or greater than 90%, equal to or greater than 95%, equal to or greater than 100%, equal to or greater than 110% or more, when compared to a control. In certain embodiments, the administration of the effective amount of VEGF accelerates wound healing between greater than 60% and 110%, when compared to a control. In certain embodiments, acceleration of wound healing is described by a Hazard ration, which is equal to or greater than 1.75, or is equal to or greater than 1.80, or is equal to or greater than 1.85, or is equal to or greater than 1.95, or is equal to or greater than 2.0, or is equal to or greater than 2.1, or is equal to or greater than 2.2, or is equal to or greater than 2.3 or more. In certain embodiments, acceleration of wound healing is described by a Hazard ration, which is between 1.75 and 2.3.

Subjects of the invention has at least one wound. The wound can be a chronic, acute or normal wound. In one embodiment, the wound being treated is a stage 1A wound. See Stages of Wounds in Table 2. A wound of the invention can optionally include an infection or ischemia, or include both an infection and ischemia. In one embodiment, the wound is a diabetic foot ulcer. In one embodiment, the wound is present on the subject for about 4 weeks or more, or about 6 weeks or more before administering the VEGF.

TABLE 2

| | Wound Grade/Depth | | | |
|---|---|---|---|---|
| Stage/Comorbidities | 0 | 1 | 2 | 3 |
| A | Pre- or post- | Superficial | Ulcer | Ulcer |
| | ulcerative lesion | ulcer not | penetrating to | penetrating |
| | completely | involving | tendon or | to bone or |
| | epithelialized | tendon, | capsule | joint |
| | | capsule, or | | |
| | | bone | | |
| B | With Infection | With Infection | With Infection | With |
| | | | | Infection |
| C | With Ischemia | With Ischemia | With Ischemia | With |
| | | | | Ischemia |
| D | With Infection | With Infection | With Infection | With |
| | and Ischemia | and Ischemia | and Ischemia | Infection |
| | | | | and |
| | | | | Ischemia |

Quantitative analysis can be used to assess wound healing, e.g., determining the % reduction in the wound area, or complete wound closure (e.g., measured by skin closure without drainage or dressing requirements). Wound area is assessed before, during, and after treatment by methods known to those in the art. For example, assessment can be determined by, e.g., quantitative planimetry (see, e.g., Robson et al., Arch. Surg 135:773-77 (2000)), photographs, physical examinations, etc. The wound area can be determined before, during and after treatment. In one embodiment, the wound area can be estimated by measuring the length, L, of the wound, the longest edge-to-edge length in, e.g., cm, and the width, W, the longest edge-to-edge width perpendicular to L in, e.g., cm, and multiplying the L×W to get the estimated surface area (cm²). The size of the wound for treatment can vary. In one embodiment of the invention, the wound area before treatment is about 0.4 cm² or more, or about 1.0 cm² or more, or between about 0.4 cm² and about 10 cm², or between about 1 cm² and about 10 cm², or between about 1 cm² and about 6.5 cm², or between about 1 cm² and about 5 cm², or more than 4.0 cm². The area can be measured before or after debridement.

VEGF

An effective amount of VEGF is administered in the methods provided herein to promote accelerated or improve wound healing. The VEGF gene family, for which VEGF is a member, is one of the key regulators of the development of the vascular system. The VEGF gene family includes VEGF-A, VEGF-B, VEGF-C, VEGF-D, VEGF-E and placental growth factor (PIGF). See, e.g., Ferrara, Role of Vascular endothelial growth factor in physiologic and pathologic angiogenesis: therapeutic implications, Sem Oncol., 29(suppl 16): 10-14 (2002); and, Veikkola and Alitalo, VEGFs receptors and angiogenesis Semin Cancer Biol. 9:211-220 (1999). VEGF-A, also known as VEGF, is a major regulator of normal angiogenesis including normal wound healing and bone healing and abnormal angiogenesis, such as vascular proliferation in tumors and ophthalmologic disorders (e.g., age-related degeneration, diabetic retinopathy). See, e.g., Ferrara, Vascular Endothelial Growth Factor: Basic Science and Clinical Progress, Endocrine Reviews 25(4): 581-611 (2004); Ferrara and Henzel, Pituitary follicular cells secrete a novel heparin-binding growth factor specific for vascular endothelial cells, Biochem, Biophys Res Comm 161:851-58 (1989); and Leung et al., Vascular endothelial growth factor is a secreted angiogenic mitogen, Science 246:1306-9 (1989). It is within the scope of the invention to also use VEGF variants having VEGF activity and agonist of the VEGF receptors, e.g., VEGFR1 and/or VEGFR2 agonists, in place of or in addition to VEGF.

Human VEGF exists as at least six isoforms (VEGF₁₂₁, VEGF₁₄₅, VEGF₁₆₅, VEGF₁₈₃, VEGF₁₈₉, and VEGF₂₀₆) that arise from alternative splicing of mRNA of a single gene organized into 8 exons located on chromosome 6 (see, e.g., Ferrara N, Davis Smyth T. Endocr Rev 18:1-22 (1997); and, Henry and Abraham, Review of Preclinical and Clinical Results with Vascular Endothelial Growth Factors for Therapeutic Angiogenesis, Current Interventional Cardiology Reports, 2:228-241 (2000)). See also, U.S. Pat. Nos. 5,332,671 and 6,899,882. In one embodiment, VEGF₁₆₅ is administered in the methods of the invention. Typically, human VEGF₁₆₅ is used (e.g., recombinant human VEGF₁₆₅). VEGF₁₆₅, the most abundant isoform, is a basic, heparin binding, dimeric covalent glycoprotein with a molecular mass of ˜45,000 daltons (Id). VEGF₁₆₅ homodimer consists of two 165 amino acid chains. The protein has two distinct domains: a receptor binding domain (residues 1-110) and a heparin binding domain (residues 110-165). The domains are stabilized by seven intramolecular disulfide bonds, and the monomers are linked by two interchain disulfide bonds to form the native homodimer. VEGF₁₂₁, lacks the heparin binding domain (see, e.g., U.S. Pat. No. 5,194,596), whereas VEGF₁₈₉ (see, e.g., U.S. Pat. Nos. 5,008,196; 5,036,003; and, 5,240,848) and VEGF₂₀₆ are sequestered in the extracellular matrix. See, e.g., Ferrara VEGF and the quest for tumor angiogenesis factors, Nature Rev. Cancer 2:795-803 (2002).

The biological effects of VEGF are mediated through high affinity tyrosine kinase receptors. Agonists of the VEGF receptors can also be used in the methods of the invention. Two VEGF receptor tyrosine kinases, VEGFR1and VEGFR2, have been identified (Shibuya et al. Oncogene 5:519-24 (1990); Matthews et al., Proc Natl Acad Sci U SA 88:9026-30 (1991); Terman et al., Oncogene 6:1677-83 (1991); Terman et al. Biochem Biophys Res Commun 187:1579-86 (1992); de Vries et al., Science 255:989-91 (1992); Millauer et al. Cell 72:83546 (1993); and, Quinn et al. Proc Natl Acad Sci USA 90:7533-7 (1993)). VEGFR1 has the highest affinity for VEGF, with a Kd of ˜10-20 pM (de Vries et al., Science 255:989-91 (1992)), and VEGFR2 has a somewhat lower affinity for VEGF, with a Kd of ˜75-125 pM (Terman et al., Oncogene 6:1677-83 (1991); Millauer et al. Cell 72:83546 (1993); and, Quinn et al. Proc Natl Acad Sci USA 90:7533-7 (1993)). A third tyrosine kinase receptor, VEGFR3 has been identified, which is involved in the regulation of lymphatic angiogenesis. VEGFR3 is a receptor for VEGF-C and VEGF-D, which can also bind VEGFR2. These receptors consist of an extracellular domain (including seven immunoglobulin-like regions, a transmembrane region) and an intracellular domain that contains elements related to the tyrosine kinase pathways. VEGF-B and PIGF binds to VEGFR1 but not VEGFR2. VEGF₁₆₅ also binds to neuropilin-1 a receptor that regulates neuronal cell guidance. When co-expressed with VEGFR2, neuropilin-1 enhances the binding of VEGF₁₆₅ to VEGFR2 and VEGF-mediated chemotaxis. Other studies have linked neuropilin 2 (NP2) to lymphatic vessel development. See, e.g., Ferrara, Vascular Endothelial Growth Factor: Basic Science and Clinical Progress, Endocrine Reviews, 254(4):581-611. After binding to VEGF-A, VEGFR2 undergoes tyrosine autophosphorylation that leads to subsequent angiogenesis, increased vascular permeability, mitogenesis, and chemotaxis.

VEGF has several biologic functions, including regulation of VEGF gene expression under hypoxic conditions (Ferrara N, Davis Smyth T. Endocr Rev 18:1-22 (1997)), mitogenic activity for micro and macrovascular endothelial cells (Ferrara N, Henzel W J. Biochem Biophys Res Commun 161:851-8 (1989); Leung et al., Science 246:1306-9 (1989); Connolly et al. J Clin Invest 84:1470-8 (1989a); Keck et al. Science 246:1309-12 (1989); Plouet et al., EMBO J 8:3801-6 (1989); Conn et al. Proc Natl Acad Sci USA 87:2628-32 (1990); and, Pepper et al., Exp Cell Res 210:298-305 (1994)), and induction of expression of plasminogen activators and collagenase (Pepper et al., Biochem Biophys Res Commun 181:902-6 (1991)). During hypoxia, hypoxia-inducing factor-1 (HIP-1) is upregulated and binds to the promoter region of the VEGF gene and activates transcription (see, e.g., Wang et al., Hypoxia-inducible factor 1 is a basic-helix-loop-helix-PAS heterodimer regulated by cellular oxygen tension, PNAS USA 92:5510 (1995)). Other agonists that can up-regulate VEGF include cytokines (IL-6) and other growth factors-including EGF, PDGF, bFGF.

VEGF is produced by a wide variety of normal cell types (e.g., keratinocytes, platelets, macrophages, fibroblasts, retinal cells, ovarian cells) throughout the body in addition to various types of solid tumors. Work done over the last several years has established a key role of vascular endothelial growth factor (VEGF) in the regulation of normal and abnormal angiogenesis (Ferrara et al. Endocr. Rev. 18:4-25 (1997)). VEGF is a necessary growth factor for normal embryonic vasculogenesis, cardiac myocyte development (see, e.g., Ferrara et al., Heterozygous embryonic lethality induced by targeted inactivation of the VEGF gene, Nature 380:439-42 (1996)), normal enchondral bone formation (see, e.g., Gerber et al., VEGF couples hypertrophic cartilage remodeling, ossification, and angiogenesis during enchondral bone formation, Nat. Med, 5:623-28 (1999)), tissue repair, and in the physiology of the female reproductive tract—follicular growth and the endocrine function of the corpus luteum are dependent on proliferation of new capillaries (see, e.g., Phillips et al., Vascular endothelial growth factor is expressed in rat corpus luteum, Endocrinology, 127:965-67 (1990)). Furthermore, VEGF has been shown to be a key mediator of neovascularization associated with tumors and intraocular disorders (Ferrara et al.). The VEGF mRNA is overexpressed by the majority of human tumors examined (Berkman et al. J Clin Invest 91:153-159 (1993); Brown et al. Human Pathol. 26:86-91 (1995); Brown et al. Cancer Res. 53:4727-4735 (1993); Mattern et al. Brit. J. Cancer. 73:931-934 (1996); and Dvorak et al. Am J. Pathol. 146:1029-1039 (1995)).

VEGF is also known as vascular permeability factor, based on its ability to induce vascular leakage in animal models. See, e.g., Senger et al., Tumor cells secrete a vascular permeability factor that promotes accumulation of ascites fluid, Science 219: 983-89 (1983). Senger and colleagues proposed that an increase in microvascular permeability to proteins is a crucial step in angiogenesis. For example, induced leakage of plasma proteins and formation of extracellular fibrin gel can be sufficient matrix for endothelial cell growth; the role of mitogenic growth factors can be to boost this process. Angiogenesis is also required to allow migration of leukocytes, growth factors, and oxygen during granulation tissue formation during wound healing.

In wound healing, VEGF plays a pivotal role in the induction of angiogenesis during cutaneous wound healing. It is a potent mitogen for dermal microvascular endothelial cells and is expressed by keratinocytes of healing wounds (See, e.g., Nissen et al., Vascular endothelial growth factor mediates angiogenic activity during the proliferative phase of wound healing, Am. J. Pathol., 152:1445-52 (1998); Corral et al., Vascular endothelial growth factor is more important than basic fibroblast growth factor during ischemic wound healing, Arch Surg., 134:200-5 (1999); Frank et al., Regulation of vascular endothelial growth factor expression in cultured keratinocytes, Implications for normal and impaired wound healing. J. Biol. Chem., 270:12607-13 (1995); Ballaun et al., Human keratinocytes express the three major splice forms of vascular endothelial growth factor, J. Invest Dermatol., 104:7-10 (1995). It also acts in a paracrine manner on dermal microvessels, leading to increased skin vascularity and granulation matrix formation. See, e.g., Corral et al., supra; and, Romano de Peppe, et al., Adenovirus-mediated VEGF165 gene transfer enhances wound healing by promoting angiogenesis in CD1 diabetic mice, Gen Ther., 9:1271-7 (2002). Deficiencies in tissue repair, e.g., wound healing, etc., are seen when VEGF levels and other angiogenic factors are altered. See, e.g., Howdieshell, et al., Antibody neutralization of vascular endothelial growth factor inhibits wound granulation tissue formation, J. Surg. Res. 96: 173-82 (2001); Street et al., Vascular endothelial growth factor stimulates bone repair by promoting angiogenesis and bone turnover, PNAS USA, 99:9656-61 (2002); Tsou et al., Retroviral delivery of dominant-negative vascular endothelial growth factor receptor type 3 to murine wounds inhibits wound angiogenesis, Wound Repair Regen., 10:222-9 (2002); Frank et al., Regulation of vascular endothelial growth factor expression in cultured keratinocytes, Implications for normal and impaired wound healing. J. Biol. Chem., 270:12607-13 (1995); and, Lerman et al., Cellular dysfunction in the diabetic fibroblast: impairment in migration, vascular endothelial growth factor production, and response to hypoxia, Am. J. Pathol., 162:303-12 (2003). In some animal models, exogenous VEGF promoted wound healing. See, e.g., Galliano et al., Topical Vascular Endothelial Growth Factor Accelerates Diabetic Wound Healing through increased angiogenesis and by Mobilizing and recruiting bone marrow-derived cells, American Journal of Pathology, 164 (6):1935-1947 (2004); Romano de Peppe S et al., Adenovirus-mediated VEGF165 gene transfer enhances wound healing by promoting angiogenesis in CD1 diabetic mice, Gen Ther 9:1271-7 (2002); and, US Pat. Appl. No. US20030180259.

As described above, there are challenges to creating protein therapies to accelerating wound healing. We describe herein a clinical trial of treating ulcers with VEGF recombinant protein therapy that results in accelerated wound healing. See example 1, herein.

Additional Agents

It is within the scope hereof to combine VEGF therapy with one or more of, e.g., good wound care therapy (e.g., GWC), other novel or conventional therapies (e.g., other members of the VEGF family, growth factors such as listed herein, nerve growth factor (NGF), positive angiogenesis factors or agents or activators, anabolic steroids, bioengineered tissue replacements (e.g., Apligraph®, Dermagraf™, etc.) hyperbaric oxygen, vacuum therapy) for enhancing the activity of VEGF, in accelerating and/or improving wound healing. See, e.g., Meier and Nanney, Emerging New Drugs for Wound Repair, Expert Opin. Emerging Drugs (2006) 11(1):23-37.

Six major growth factor families (EGF, FGF, IGF, PDGF, TGF, and VEGF) are associated with wound healing. See, e.g., Nagai and Embil, Becaplermin: recombinant platelet derived growth factor, a new treatment for healing diabetic foot ulcers, Exprt Opin Biol. Ther 2:211-18 (2002). Examples of such growth factors include platelet derived growth factor (PDGF-A, PDGF-B, PDGF-C, and PDGF-D), insulin-like growth factor I and II (IGF-I and IGF-II), acidic and basic fibroblast growth factor (aFGF and bFGF), alpha and beta transforming growth factor (TGF-α and TGF-β (e.g., TGF-beta 1, TGF beta 2, TGF beta 3)), epidermal growth factor (EGF), and others. See Id. These growth factors stimulate mitosis of one or more of the cells involved in wound healing and can be combined with VEGF.

Other positive angiogenesis agents that can be combined with VEGF include, but are not limited to, e.g., HGF, TNF-α, angiogenin, IL-8, etc. (Folkman et al. J. Biol. Chem. 267:10931-10934 (1992); Klagsbrun et al. Annu. Rev. Physiol. 53:217-239 (1991)), angiogenesis activators in Table 3, angiogenesis factors and agents described herein, etc.

TABLE 3
Examples of Angiogenesis Activators
Angiogenesis

Angiopoietins 1 and 2
Tie-2
Alpha-5 integrins
Matrix metalloproteinases
Nitric oxide (NO)
COX-2
TGFbeta and receptors
VEGF and receptors

In addition, the following agents can also be combined with VEGF wound healing treatments, e.g., Platelet-derived growth factor (PDGF) (e.g., Becaplermin (rhPDGF-BB) such as Regranex®; Johnson & Johnson (see, e.g., U.S. Pat. Nos. 5,457,093; 5,705,485; and, 5,427,778; Perry, B H et al., A meta-analytic approach to an integrated summary of efficacy: a case study of becamplemin gel., Cont. Clin. Trials 23:389-408 (2002)), adenosine-A2A receptor agonists (e.g., MRE0094 (King Pharmaceuticals)); keratinocyte growth factor (KGF-2, repifermin (Human Genome Sciences)); lactoferrin (LF) (Agennix, Inc.,); thymosine beta-4 (Tβ4 (ReGeneRx Biopharmaceuticals)); thrombin-derived activating receptor peptide (TP508; Chrysalin® (Chrysalis Biotechnology, Inc.)); adenoviral vector encoding platelet-derived growth factor (PDGF-B) (GAM501) (Selective Genetics); autologous bone marrow stem cells (BMSC) (see, e.g., Badiavas & Falanga, Treatment of chronic wounds with bone marrow-derived cells, Arch Dermatol, 139:510-16 (2003); and, engineered living tissue grafts (e.g., Apligraf, etc.). Antibiotic and antiseptic ulcer agents can also be combined with VEGF administration. VEGF administration can also be administered along with immunosuppressive treatment (e.g., corticosteroids, radiation therapy, chemotherapy) or cancer treatment.

It is not necessary that such cotreatment agents or procedures be included per se in the compositions of this invention, although this will be convenient where such agents are proteinaceous. Such admixtures are suitably administered in the same manner and for the same purposes as the VEGF used alone. The useful molar ratio of VEGF to such secondary therapeutic factors is typically 1:0.1-10, with about equimolar amounts in one embodiment of the invention being used.

Dosage and Administration

Dosages and desired drug concentrations of pharmaceutical compositions of the invention may vary depending on the particular use envisioned. The determination of the appropriate dosage or route of administration is well within the skill of an ordinary physician. Animal experiments can provide reliable guidance for the determination of effective doses for human therapy. Interspecies scaling of effective doses can be performed following the principles laid down by Mordenti, J. and Chappell, W. The use of interspecies scaling in toxicokinetics In Toxicokinetics and New Drug Development, Yacobii et al., Eds., Pergamon Press, New York 1989, pp. 42-96. Examples of dose-response curves for VEGF administered animal wound models can be see in FIG. 2, which is a dose response curve for VEGF administered to rabbit ischemic ear wounds. FIG. 3 is a dose-response curve for VEGF administered to diabetic mouse wound. For the prevention or treatment of disease or type of wound, the appropriate dosage of VEGF will depend on the type of disease to be treated, as defined above, the severity and course of the disease, whether the agent is administered for preventive or therapeutic purposes, previous therapy, the patient's clinical history and response to the agent, and the discretion of the attending physician. VEGF will be formulated and dosed in a fashion consistent with good medical practice taking into account the specific disorder to be treated, the condition of the individual patient, the site of delivery of the VEGF, the method of administration, and other factors known to practitioner.

The dosage to be employed is dependent upon the factors described herein. In certain embodiments of the invention, depending on the type and severity of the condition of the subject, about 1 μg/kg to 50 mg/kg (e.g. 0.1-20 mg/kg) of VEGF and/or an additional agent, is a candidate dosage for administration to the patient, whether, for example, by one or more separate administrations, or by continuous application. Guidance as to particular dosages and methods of delivery is provided in the literature. In one embodiment, the effective amount of VEGF administered is about 20 μg/cm² to about 250 μg/cm². In certain embodiments, the effective amount administered is about 24 μg/cm², or 24 μg/cm². In certain embodiments, the effective amount administered is about 72 μg/cm², or 72 μg/cm². In certain embodiments, the effective amount administered is about 216 μg/cm², or 216 μg/cm². In one embodiment, the effective amount of VEGF administered is 20 μg/cm² to 250 μg/cm². In certain embodiments, the effective amount administered is about 24 μg/cm² to about 216 μg/cm², or 24 μg/cm² to 216 μg/cm^{2.} In certain embodiments, the effective amount administered is about 24 μg/cm² to about 72 μg/cm², or 24 μg/cm² to 72 μg/cm². In certain embodiments, the effective amount administered is about 72 μg/cm² to about 216 μg/cm², or 72 μg/cm² to 216 μg/cm². In certain embodiments, the effective amount administered is about 216 μg/cm² to about 250 μg/cm², or 216 μg/cm² to 250 μg/cm². The agent is suitably administered to the subject over a series of treatments or at one time.

For repeated administrations over several days or longer, depending on the condition, the treatment is sustained until a desired suppression of disease symptoms occurs, e.g., complete closure of the wound, or reduction in wound area. However, other dosage regimens may be useful. Typically, the clinician will administered a molecule(s) of the invention until a dosage(s) is reached that provides the required biological effect. The administration of the effective amount of VEGF can be daily or optionally a few times a week, e.g., at least twice a week, or at least three times a week, or at least four times a week, or at least five times a week, or at least six times a week. In one embodiment, the VEGF is administered at least for six weeks, or at least about twelve weeks or until complete wound closure (e.g., which can be determined by skin closure without drainage or dressing requirements). In certain aspects of the invention, the VEGF is administered for less than 20 weeks. The progress of the therapy of the invention is easily monitored by conventional techniques and assays.

The therapeutic composition of the invention is typically administered topically to the subject. In one embodiment of the invention, the VEGF is in a formulation of a topical gel, e.g., in a pre-filed syringe or container. In certain embodiments, an additional therapeutic agent is also administered topically. Other routes of administration of VEGF and/or additional therapeutic agents, can also be optionally used, e.g., administered by any suitable means, including but not limited to, parenteral, subcutaneous, intraperitoneal, intrapulmonary, intracerobrospinal, subcutaneous, intra-articular, intrasynovial, intrathecal, oral, and intranasal administration. Parenteral infusions include intramuscular, intravenous, intraarterial, intraperitoneal, or subcutaneous administration.

As described herein, VEGF can be combined with one or more additional therapeutic agents or procedures. The combined administration includes coadministration, using separate formulations or a single pharmaceutical formulation, and consecutive administration in either order. Use of multiple agents is also included in the invention. For example, VEGF may precede, follow, alternate with administration of the additional therapeutic agent, or may be given simultaneously therewith. In one embodiment, there is a time period while both (or all) active agents simultaneously exert their biological activities. In a combination therapy regimen, the compositions of the invention are administered in a therapeutically effective amount or a therapeutically synergistic amount. As used herein, a therapeutically effective amount is such that co-administration of VEGF and one or more other therapeutic agents, or administration of a procedure, results in reduction or inhibition of the targeting disease or condition. A therapeutically synergistic amount is that amount of VEGF and one or more other therapeutic agents, e.g., described herein, necessary to synergistically or significantly accelerate and/or improve wound healing.

Pharmaceutical Compositions

Therapeutic formulations of molecules of the invention, e.g., VEGF or additional therapeutic agents combined with VEGF, used in accordance with the invention are prepared for storage by mixing a molecule, e.g., a polypeptide, having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In certain embodiments, the formulations to be used for in vivo administration are sterile. This is readily accomplished by filtration through sterile filtration membranes. The VEGF can be stored in lyophilized form or as an aqueous solution or gel form. The pH of the VEGF preparations can be about from 5 to 9, although higher or lower pH values may also be appropriate in certain instances. It will be understood that use of certain of the excipients, carriers, or stabilizers can result in the formation of salts of the VEGF.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). See also Johnson et al., Nat. Med., 2:795-799 (1996); Yasuda, Biomed. Ther., 27:1221-1223 (1993); Hora et al., Bio/Technology, 8:755-758 (1990); Cleland, Design and Production of Single Immunization Vaccines Using Polylactide Polyglycolide Microsphere Systems, in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010.

Typically for wound healing, VEGF is formulated for site-specific delivery. When applied topically, the VEGF is suitably combined with other ingredients, such as carriers and/or adjuvants. There are no limitations on the nature of such other ingredients, except that they must be pharmaceutically acceptable and efficacious for their intended administration, and cannot degrade the activity of the active ingredients of the composition. Examples of suitable vehicles include ointments, creams, gels, sprays, or suspensions, with or without purified collagen. The compositions also may be impregnated into sterile dressings, transdermal patches, plasters, and bandages, optionally in liquid or semi-liquid form. An oxidized regenerated cellulose/collagen matrices can also be used, e.g., Promogran™ Matrix Wound Dressing or Promogran Prisma Matrix™.

Sustained-release preparations may be prepared. Suitable examples of sustained-release preparations include semipermeable matrices of solid hydrophobic polymers containing a polypeptide of the invention, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), poly-lactic-coglycolic acid (PLGA) polymer, and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

For obtaining a gel formulation, the VEGF formulated in a liquid composition may be mixed with an effective amount of a water-soluble polysaccharide or synthetic polymer such as polyethylene glycol to form a gel of the proper viscosity to be applied topically. The polysaccharide that may be used includes, for example, cellulose derivatives such as etherified cellulose derivatives, including alkyl celluloses, hydroxyalkyl celluloses, and alkylhydroxyalkyl celluloses, for example, methylcellulose, hydroxyethyl cellulose, carboxymethyl cellulose, hydroxypropyl methylcellulose, and hydroxypropyl cellulose; starch and fractionated starch; agar; alginic acid and alginates; gum arabic; pullullan; agarose; carrageenan; dextrans; dextrins; fructans; inulin; mannans; xylans; arabinans; chitosans; glycogens; glucans, and synthetic biopolymers; as well as gums such as xanthan gum; guar gum; locust bean gum; gum arabic; tragacanth gum; and karaya gum; and derivatives and mixtures thereof. In one embodiment of the invention, the gelling agent herein is one that is, e.g., inert to biological systems, nontoxic, simple to prepare, and/or not too runny or viscous, and will not destabilize the VEGF held within it.

In certain embodiments of the invention, the polysaccharide is an etherified cellulose derivative, in another embodiment one that is well defined, purified, and listed in USP, e.g., methylcellulose and the hydroxyalkyl cellulose derivatives, such as hydroxypropyl cellulose, hydroxyethyl cellulose, and hydroxypropyl methylcellulose. In one embodiment, methylcellulose is the polysaccharide.

The polyethylene glycol useful for gelling is typically a mixture of low and high molecular weight polyethylene glycols to obtain the proper viscosity. For example, a mixture of a polyethylene glycol of molecular weight 400-600 with one of molecular weight 1500 would be effective for this purpose when mixed in the proper ratio to obtain a paste.

The term “water soluble” as applied to the polysaccharides and polyethylene glycols is meant to include colloidal solutions and dispersions. In general, the solubility of the cellulose derivatives is determined by the degree of substitution of ether groups, and the stabilizing derivatives useful herein should have a sufficient quantity of such ether groups per anhydroglucose unit in the cellulose chain to render the derivatives water soluble. A degree of ether substitution of at least 0.35 ether groups per anhydroglucose unit is generally sufficient. Additionally, the cellulose derivatives may be in the form of alkali metal salts, for example, the Li, Na, K, or Cs salts.

If methylcellulose is employed in the gel, e.g., it comprises about 2-5%, or about 3%, or about 4% or about 5%, of the gel and the VEGF is present in an amount of about 100-2000 μg per ml of gel.

VEGF and/or an additional agent can also be administered to the wound by gene therapy. Gene therapy refers to therapy performed by the administration of a nucleic acid to a subject. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. The oligonucleotides can be modified to enhance their uptake, e.g. by substituting their negatively charged phosphodiester groups by uncharged groups. For general reviews of the methods of gene therapy, see, for example, Goldspiel et al. Clinical Pharmacy 12:488-505 (1993); Wu and Wu Biotherapy 3:87-95 (1991); Tolstoshev Ann. Rev. Pharmacol. Toxicol. 32:573-596 (1993); Mulligan Science 260:926-932 (1993); Morgan and Anderson Ann. Rev. Biochem. 62:191-217 (1993); and May TIBTECH 11:155-215 (1993). Methods commonly known in the art of recombinant DNA technology which can be used are described in Ausubel et al. eds. (1993) Current Protocols in Molecular Biology, John Wiley & Sons, NY; and Kriegler (1990) Gene Transfer and Expression, A Laboratory Manual, Stockton Press, NY.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11, 205-210 (1993)). For example, in vivo nucleic acid transfer techniques include transfection with viral vectors (such as adenovirus, Herpes simplex I virus, lentivirus, retrovirus, or adeno-associated virus) and lipid-based systems (useful lipids for lipid-mediated transfer of the gene are DOTMA, DOPE and DC-Chol, for example). Examples of using viral vectors in gene therapy can be found in Clowes et al. J. Clin. Invest. 93:644-651 (1994); Kiem et al. Blood 83:1467-1473 (1994); Salmons and Gunzberg Human Gene Therapy 4:129-141 (1993); Grossman and Wilson Curr. Opin. in Genetics and Devel. 3:110-114 (1993); Bout et al. Human Gene Therapy 5:3-10 (1994); Rosenfeld et al. Science 252:431-434 (1991); Rosenfeld et al. Cell 68:143-155 (1992); Mastrangeli et al. J. Clin. Invest. 91:225-234 (1993); and Walsh et al. Proc. Soc. Exp. Biol. Med. 204:289-300 (1993).

In some situations it is desirable to provide the nucleic acid source with an agent that targets the cells of a wound, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). For review of gene marking and gene therapy protocols see Anderson et al., Science 256, 808-813 (1992).

Covalent Modifications to Polypeptides of the Invention

Covalent modifications of a polypeptide of the invention, e.g., VEGF or other additional therapeutic polypeptide agents combined with VEGF, are included within the scope of this invention. They may be made by chemical synthesis or by enzymatic or chemical cleavage of the polypeptide, if applicable. Other types of covalent modifications of the polypeptide are introduced into the molecule by reacting targeted amino acid residues of the polypeptide with an organic derivatizing agent that is capable of reacting with selected side chains or the N- or C-terminal residues, or by incorporating a modified amino acid or unnatural amino acid into the growing polypeptide chain, e.g., Ellman et al. Meth. Enzym. 202:301-336 (1991); Noren et al. Science 244:182 (1989); and, & US Patent applications 20030108885 and 20030082575.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is typically performed in 0.1 M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing α-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKₐ of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay.

Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues, respectively. These residues are deamidated under neutral or basic conditions. The deamidated form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Another type of covalent modification involves chemically or enzymatically coupling glycosides to a polypeptide of the invention. These procedures are advantageous in that they do not require production of the polypeptide in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 published 11 Sep. 1987, and in Aplin and Wriston, CRC Crit. Rev. Biochem., pp. 259-306 (1981).

Removal of any carbohydrate moieties present on a polypeptide of the invention may be accomplished chemically or enzymatically. Chemical deglycosylation requires exposure of the polypeptide to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin, et al. Arch. Biochem. Biophys. 259:52 (1987) and by Edge et al. Anal. Biochem., 118:131 (1981). Enzymatic cleavage of carbohydrate moieties, e.g., on antibodies, can be achieved by the use of a variety of endo- and exo-glycosidases as described by Thotakura et al. Meth. Enzymol. 138:350 (1987).

Another type of covalent modification of a polypeptide of the invention comprises linking the polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Vectors, Host Cells and Recombinant Methods

The polypeptides of the invention can be produced recombinantly, using techniques and materials readily obtainable.

For recombinant production of a polypeptide of the invention, e.g., VEGF or additional therapeutic polypeptide agents combined with VEGF, the nucleic acid encoding it is isolated and inserted into a replicable vector for further cloning (amplification of the DNA) or for expression. Many vectors are available. The vector components generally include, but are not limited to, one or more of the following: control sequences, a signal sequence, an origin of replication, one or more marker genes, an enhancer element, a promoter, and a transcription termination sequence.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

DNA encoding the polypeptide of the invention is readily isolated and/or sequenced using conventional procedures. For example, a DNA encoding VEGF is isolated and sequenced, e.g., by using oligonucleotide probes that are capable of binding specifically to the gene encoding VEGF. An “isolated” nucleic acid molecule is a nucleic acid molecule that is identified and separated from at least one contaminant nucleic acid molecule with which it is ordinarily associated in the natural source of the polypeptide nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the polypeptide where, for example, the nucleic acid molecule is in a chromosomal location different from that of natural cells.

Signal Sequence Component

Polypeptides of the invention may be produced recombinantly not only directly, but also as a fusion polypeptide with a heterologous polypeptide, which is typically a signal sequence or other polypeptide having a specific cleavage site at the N-terminus of the mature protein or polypeptide. The heterologous signal sequence selected typically is one that is recognized and processed (i.e., cleaved by a signal peptidase) by the host cell. For prokaryotic host cells that do not recognize and process the native polypeptide signal sequence, the signal sequence is substituted by a prokaryotic signal sequence selected, for example, from the group of the alkaline phosphatase, penicillinase, lpp, or heat-stable enterotoxin II leaders. For yeast secretion the native signal sequence may be substituted by, e.g., the yeast invertase leader, a factor leader (including Saccharomyces and Kluyveromyces α-factor leaders), or acid phosphatase leader, the C. albicans glucoamylase leader, or the signal described in WO 90/13646. In mammalian cell expression, mammalian signal sequences as well as viral secretory leaders, for example, the herpes simplex gD signal, are available.

The DNA for such precursor region is ligated in reading frame to DNA encoding the polypeptide of the invention.

Origin of Replication Component

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the nucleic acid, such as DHFR, thymidine kinase, metallothionein-I and -II, typically primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity.

Alternatively, host cells (particularly wild-type hosts that contain endogenous DHFR) transformed or co-transformed with DNA sequences encoding a polypeptide of the invention, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid Yrp7 (Stinchcomb et al., Nature, 282:39 (1979)). The trp1 gene provides a selection marker for a mutant strain of yeast lacking the ability to grow in tryptophan, for example, ATCC No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 9:968-975 (1991).

Promotor Component

Expression and cloning vectors usually contain a promoter that is recognized by the host organism and is operably linked to a nucleic acid encoding a polypeptide of the invention. Promoters suitable for use with prokaryotic hosts include the phoA promoter, β-lactamase and lactose promoter systems, alkaline phosphatase, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the polypeptide of the invention.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldyhyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldyhyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Transcription of polypeptides of the invention from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and typically Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the rous sarcoma virus long terminal repeat can be used as the promoter.

Enhancer Element Component

Transcription of a DNA encoding a polypeptide of this invention by higher eukaryotes is often increased by inserting an enhancer sequence into the vector. Many enhancer sequences are now known from mammalian genes (globin, elastase, albumin, α-fetoprotein, and insulin). Typically, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide-encoding sequence, but is typically located at a site 5′ from the promoter.

Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the polypeptide of the invention. One useful transcription termination component is the bovine growth hormone polyadenylation region. See WO94/11026 and the expression vector disclosed therein.

Selection and Transformation of Host Cells

Suitable host cells for cloning or expressing DNA encoding the polypeptides of the invention in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. Typically, the E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for polypeptide of the invention-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated polypeptides of the invention are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR(CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (WI 38, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for polypeptide of the invention production and cultured in conventional nutrient media modified as appropriate for inducing promoters, selecting transformants, or amplifying the genes encoding the desired sequences.

Culturing the Host Cells

The host cells used to produce polypeptides of the invention may be cultured in a variety of media. Commercially available media such as Ham's F10 (Sigma), Minimal Essential Medium ((MEM), (Sigma), RPMI-1640 (Sigma), Dulbecco's Modified Eagle's Medium ((DMEM), Sigma), normal growth media for hepatocytes (Cambrex), growth media for pre-adipocytes (Cambrex), etc. are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Polypeptide Purification

When using recombinant techniques, a polypeptide of the invention, e.g., VEGF or additional therapeutic polypeptide agent that is combined with VEGF, can be produced intracellularly, in the periplasmic space, or directly secreted into the medium. Polypeptides of the invention may be recovered and/or isolated from culture medium or from host cell lysates. An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In certain embodiments, the polypeptide will be purified (1) to greater than 95% by weight of polypeptide as determined by the Lowry method, or more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue, or silver stain. Isolated polypeptide includes the polypeptide in situ within recombinant cells since at least one component of the polypeptide's natural environment will not be present. Ordinarily, however, isolated polypeptide will be prepared by at least one purification step.

Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular polypeptide of the invention produced. If membrane-bound, polypeptides of the invention can be released from the membrane using a suitable detergent solution (e.g. Triton-X 100) or by enzymatic cleavage. Cells employed in expression of a polypeptide of the invention can be disrupted by various physical or chemical means, such as freeze-thaw cycling, sonication, mechanical disruption, or cell lysing agents. The following procedures are exemplary of suitable purification procedures: by fractionation on an ion-exchange column; ethanol precipitation; reverse phase HPLC; chromatography on silica, chromatography on heparin SEPHAROSE™ chromatography on an anion or cation exchange resin (such as a polyaspartic acid column, DEAE, etc.); chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of polypeptides of the invention.

For example, a VEGF composition prepared from the cells can be purified using, for example, heparin chromatography, gel electrophoresis, and dialysis. Other techniques for protein purification are also available.

Articles of Manufacture

In another embodiment of the invention, an article of manufacture containing materials useful for the methods and treatment of wounds described above is provided. The article of manufacture comprises a container, a label and a package insert. Suitable containers include, for example, bottles, vials, syringes, dressings, bandages etc. The containers may be formed from a variety of materials such as glass, plastic, nylon, cotton, polyester, etc. The container holds a composition which is effective for treating the condition and may have a sterile access port or may be a tube with multiple dosages or may be a syringe with indications of measured amounts of active agent. At least one active agent in the composition is included in the container. The label on, or associated with, the container indicates that the composition is used for accelerating or improving wound healing. The article of manufacture may further comprise a second container comprising a pharmaceutically-acceptable buffer, such as normal saline, phosphate-buffered saline, Ringer's solution and dextrose solution, or gel solution. It may further include other materials desirable from a commercial and user standpoint, including other buffers, diluents, filters, dressings, bandages, applicators, gauze, barriers, semi-permeable barriers, tongue depressors, needles, and syringes. Optionally, a set of instructions, generally written instructions, is included, which relates to the use and dosage of VEGF for administering to the wound described herein. The instructions included with the kit generally include information as to dosage, dosing schedule, and route of administration for the treatment the disorder. The containers of VEGF may be unit doses, bulk packages (e.g., multi-dose packages), or sub-unit doses.

The specification is considered to be sufficient to enable one skilled in the art to practice the invention. Indeed, various modifications of the invention in addition to those shown and described herein will become apparent to those skilled in the art from the foregoing description and fall within the scope of the appended claims.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

### Example 1

### Topical VEGF in wound healing VGF2763g Clinical Trial

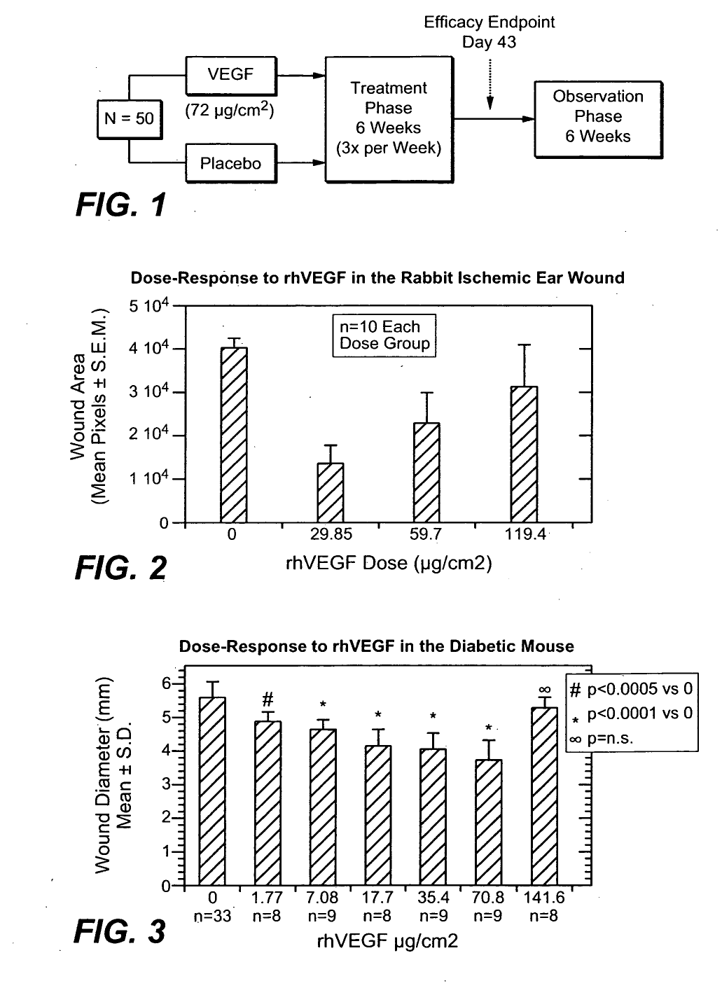

A double-blind (e.g., pharmacist unblinded, MD blinded and patient blinded) clinical trial was performed to determine if application of topical VEGF could promote wound healing in human subjects with diabetic ulcerations. See Table 4 for a chart of baseline disease characteristics of the patients in the study for administering rhVEGF (as referred herein as “Telbermin”) for the treatment of diabetic wounds. The design of the study is indicated in FIG. 1.

TABLE 4
Baseline Disease Characteristics

| | Placebo (N = 26) | | Telbermin (N = 29) | |
|---|---|---|---|---|
| Mean Age, y (range) | 59.3 | (38-81) | 59.5 | (42-74) |
| Mean Glucose, | 225.8 | (77-465) | 179.1 | (29-593) |
| mg/dL (range)* | | | | |
| Mean HbA1C, % (range)† | 8.4 | (5.5-13.6) | 8.3 | (5.6-13.6) |
| Ulcer Debrided | | | | |
| at Screening, N (%) | | | | |
| Yes | 21 | (80.8) | 27 | (93.1) |
| No | 5 | (19.2) | 2 | (6.9) |
| Mean Ulcer Area, cm² (range) | | | | |
| Length × Width at Screening | 1.85 | (1.08-2.90) | 1.92 | (0.96-4.08) |
| Planimetry at Screening‡ | 1.14 | (0.50-2.24) | 1.35 | (0.59-3.51) |
| Planimetry at Day 1 | 1.05 | (0.62-2.34) | 1.15 | (0.44-2.97) |

*One placebo-treated subject was excluded from summary because of a missing glucose value.
†One telbermin-treated subject was excluded from summary because of a missing HbA1Cvalue.
‡Two placebo-treated subjects did not have baseline planimetry assessments.

24 of the placebo treated patients completed the treatment phase and 22 completed the observation phase. 27 of the telbermin (topical recombinant VEGF) treated patients completed the treatment phase and 22 completed the observation phase. Patients with diabetes mellitus I or II (controlled, glycosylated hemoglobin Alc (HbA1c)≦12%) with debrided ulcer area of ≧0.4 cm² and ≦4.0 cm² at day 1 that were superficial wounds (e.g., at UT stage 1a (no bone, muscle, tendon), see Table 2) were treated with either VEGF or a placebo 3 times a week for 6 weeks (18 doses total). Treatment was every 48 hours (+/−24 hours) but no more than 3 doses per week. The amount of VEGF per treatment was 72 μg/cm². The VEGF was prepared on site. 0.22 ml of VEGF (5 mg/ml) or the Placebo (buffer vehicle) was removed from the vial and added to about a 5% methylcellulose (e.g., 4.7%) (e.g., Methocel A4M premium methylcellulose (The Dow Chemical Company; Midland, Mich.) formulation in 5 mM succinate buffer, pH 5.0. The VEGF or Placebo and the gel were mixed for 2 hours, which increased viscosity and reduced the loss of dosing material when applied. A final 0.06% VEGF gel (final gel 3% methylcellulose) in 5 mM, pH 5.0 succinate buffer (with, e.g., at VEGF 1.8 mg/ml, 0.0036% polysorbate 20 and 100 mM trehalose dehydrate) was the final dosing material. The final dosing material was applied with a 1.0 ml tuberculin syringe, e.g., filled with 0.6 mL of final dosing material.

Typically, the ulcer was a chronic ulcer. The ulcer duration was greater than or equal to 4 weeks to less than 6 months before treatment. There was no active infection and the subject had a perfused limb: ankle-brachial index (ABI)≧0.6 and less than or equal to 1.2 on the study foot. During the treatment the two groups VEGF or placebo had good wound care practice and weekly assessments, e.g., physical examinations, planimetric tracings and/or 35-mm photographs (e.g., Food and Drug Administration (FDA) Guidance for Industry 2000, Chronic Cutaneous Ulcer and Burn Wounds-Developing Products for Treatment, June 2000).

The endpoints to be addressed were incidence of complete wound closure, which included skin closure without drainage or dressing requirements, (e.g., assessed, e.g., 3 months following closure) and accelerated wound healing, where the rate reflects a clinically meaningful diminution of time until, e.g., complete closure occurs, and a time to event analysis (e.g., time to complete closure). Primary efficacy endpoint was percent reduction in total ulcer surface area at day 43 (up to Day 49) from baseline was determined by quantitative analysis of planimetric tracings of the ulcer. Secondary efficacy endpoints included: percent reduction in total ulcer surface area at Day 29 and Day 84 compared with baseline (e.g., Day 1 value), incidence of complete ulcer healings at Days 29, 43, and 84, time (e.g., days) to complete healing of ulcer, time (e.g., days) to recurrence of ulcer formation for subject with complete ulcer healing prior to end of treatment, incidence of increased total ulcer surface area (>15%) compared with baseline, incidence of advancing ulcer stage (e.g.,>UT 1a), and microcirculatory perfusion of the ulcer bed at Days 1, 8, 22, and 43.

Safety issues that were monitored involved clinically-significant hypotension (e.g., defined as a drop of ≧35 mmHg in systolic blood pressure relative to predose at 60 minutes after the application of each dose of study drug during the first treatment week (days 1, 3 and 5), clinically-significant ulcer infection (e.g., defined by increased discharge and malodorous exudates from the ulcer, fever (temperature of ≧38.6° C.), and a white blood cell (WBC) count of >10,000 μL), production of anti-VEGF antibodies, etc. Blood pressure was measured prior to each does and 60 minutes after each dose during the first week.

Total volume of gel applied for each treatment is 0.12-0.48 mL (72 μg-288 μg VEGF). The amount of gel applied was based upon wound measurements (L×W), where L is the longest edge-to-edge length in cm and W is the longest edge-to-edge width perpendicular to L in cm (L×W=estimated surface area (cm²)). For example, the gel is applied by using a sterile tongue depressor, where the total amount of gel applied over entire surface of ulcer, was at a thickness of 1/16″. The wound is covered with sterile, semipermeable barrier (e.g., adaptec film dressing) and wrapped with cotton gauze (e.g., Kerlix) wrap. At the next treatment, the dressing is removed and the ulcer gently irrigated with sterile normal saline. Ulcer surface is measured again, the appropriate dose of gel is applied and the ulcer is redressed.

Results: Topical VEGF appears to be safe and well-tolerated. Incidence of adverse events was comparable between treatment groups (telbermin and placebo groups). None of the adverse events or serious adverse events observed were attributed to the study drug. Two patients discontinued the study due to serious adverse events (1 in the telbermin group→ infected skin ulcer; 1 in the placebo group→localized infection). There was one patient in the telbermin group who died 4 days following the last treatment, but the death was not attributed to the study drug. No cases of clinically significant hypotension were observed in either treatment group.

Data suggests evidence of biological activity. No safety signals were observed in a trial that had small ulcer sizes that were UT stage 1a. See Table 5 for a summary of the results for median % reduction in wound area, % of subjects with complete healing and time to healing. In diabetic subjects treated with VEGF for 6 weeks at 3 times per week, the population of subjects showed a 14-25% improvement in complete wound healing after 6 weeks with VEGF compared to placebo. The trial showed that VEGF had acceleration of healing of ˜75-100% faster than placebo. See Table 6, which illustrates the time to first complete ulcer healing in patients treated with Telbermin (rhVEGF) or placebo. Time for complete healing of the ulcer was accelerated in patients treated with VEGF, e.g., time to complete healing (25^{th} percentile) was 32.5 days verses 43.0 days.

TABLE 5

| | | Median % Reduction | % Subjects with | |
|---|---|---|---|---|
| | | in Wound Area (total | Complete Healing | Time to Healing |
| | | ulcer surface) | (VEGF vs. | (VEGF vs. |
| | | (VEGF vs. Placebo)²* | Placebo)³* | Placebo)⁴* |
| Day 43 | Safety | 95% vs. 85% (p = 0.67) | 41% vs. 27% | HR 1.75 (p = 0.18) |
| (Week 6) | Evaluable | | (p = 0.39) | |
| | Efficacy¹ | 100% vs. 88% (p = 0.17) | 52% vs. 27% | HR 1.98 (p = 0.12) |
| | Evaluable | | (p = 0.13) | |
| Day 84 | Safety | 100% vs. 92% (p = 0.49) | 52% vs. 35% | HR 1.87 (p = 0.13) |
| (WK 12) | Evaluable | | (p = 0.28) | |
| | Efficacy¹ | 100% vs. 93% (p = 0.05) | 71% vs. 38% | HR 2.10 (p = 0.08) |
| | Evaluable | | (p = 0.06) | |

¹Efficacy evaluable subjects (specified prior to unblinding): Major protocol violators removed Subjects missing 3 consecutive doses-censored at last available dosing No LOCF (missing data not imputed)
²p-value: Wilcoxon rank-sum test
³p-value: Fisher's exact test.
⁴p-value: Log-Rank test
*assessment method-quantitative planimetric analysis

TABLE 6

| Time to Complete Healing* | Placebo(N = 26) | Telbermin (N = 29) |
|---|---|---|
| 25^{th} percentile, day | 43.0 | 32.5 |
| 50^{th} percentile, day | ND | 58.0 |

ND = Not detectable
*Estimated using the Kaplan Meier method.

For subjects who achieved complete ulcer healing, recurrence of ulcer formation was assessed between the time of first complete ulcer healing and the time of study completion or discontinuation. Of the safety evaluable subjects who achieved complete ulcer healing, 26.7% of the telbermin-treated subjects (4 of 15) and 33.3% of the placebo-treated subjects (3 of 9) had a recurrence of ulcer formation (log−rank p−value=0.57). The hazard ratio for recurrence of ulcer formation for telbermin-treated subjects compared with placebo-treated subjects was 0.63 (95% CI: 0.13, 3.15).

### Example 2

### Topical VEGF in Wound Healing

Subjects, e.g., patients with diabetes mellitus I or II, with an estimated ulcer area after sharp debridement of, e.g.,≧1.0 cm² and ≦6.5 cm² at the start of treatment, are treated with topical recombinant VEGF (e.g., gel formulation) daily for 12 weeks (for a total of up to 84 doses) total or until complete wound closure (e.g., skin closure without drainage or dressing requirements), which ever comes earlier. Subjects can be observed for 12 weeks or more after the treatment phase. Subjects receive either 24 μg/cm², 72 μg/cm², or 216 μg/cm² VEGF in each daily treatment. The ulcer surface area (cm²) is estimated, e.g., by the length (L(cm)) is the longest edge-to-edge measurement of the ulcer and the width (W(cm)) is taken from a perpendicular axis to the length at the longest edge-to-edge measurement. The estimated surface area is then L×W. Treatment can be assessed by measurement of the perimeter of the ulcer area via tracings, planimetric analysis tracings of the ulcer margin, photographs, physical examinations, etc. The VEGF applied will be 1.8, 0.6 and 0.2 mg/ml of VEGF, 3% methylcellulose (e.g., Methocel A4M premium methylcellulose (The Dow Chemical Company; Midland, Mich.), in 5 mM, pH 5.0 succinate buffer (with, e.g., at VEGF 1.8 mg/ml, 0.0036% polysorbate 20 and 100 mM trehalose dehydrate).

The specification is considered to be sufficient to enable one skilled in the art to practice the invention. It is understood that the examples and embodiments described herein are for illustrative purposes only. Indeed, various modifications of the invention in addition to those shown and described herein will become apparent to those skilled in the art from the foregoing description and fall within the scope of the appended claims.

We claim:

1. A method of accelerating wound healing in a subject, the method comprising:
administering an effective amount of VEGF to a wound, wherein the administration of the effective amount of VEGF accelerates wound healing greater than 60% when compared a control.

2. The method of claim 1, wherein the acceleration of wound healing is equal to or greater than 74% when compared to the control.

3. The method of claim 1, wherein the acceleration of wound healing is assessed by % reduction in wound area.

4. The method of claim 3, wherein the wound area is about 0.4 cm² or more before treatment.

5. The method of claim 3, wherein the wound area is about 1.0 cm² or more before treatment.

6. The method of claim 1, wherein the acceleration of wound healing is assessed by rate of complete wound healing

7. The method of claim 1, wherein the wound is a diabetic foot ulcer.

8. The method of claim 1, wherein the effective amount of VEGF is administered at least three times a week.

9. The method of claim 1, wherein the effective amount of VEGF is administered at least for six weeks.

10. The method of claim 1, wherein the effective amount of VEGF is administered until there is complete wound closure.

11. The method of claim 1, wherein the VEGF is VEGF₁₆₅.

12. The method of claim 1 or 11, wherein the VEGF is recombinant human VEGF.

13. The method of claim 1, wherein the administration is topical.

14. The method of claim 1, wherein the VEGF is in a formulation for topical administration.

15. The method of claim 1, wherein the wound is a chronic wound.

16. The method of claim 1, wherein the wound is a pressure ulcer, a decubitus ulcer, a venous ulcer, a burn, a surgical wound, or a normal wound.

17. The method of claim 1, wherein the subject is undergoing or has undergone a treatment, wherein the treatment delays or provides ineffective wound healing.

18. The method of claim 1, wherein the subject has a secondary condition, wherein the secondary conditions delays or provides ineffective wound healing.

19. The method of claim 18, wherein the secondary condition is diabetes.

20. The method of claim 1, wherein the effective amount of VEGF is about 20 μg/cm² to about 250 μg/cm².

21. The method of claim 20, wherein the effective amount of VEGF is about 24 μg/cm².

22. The method of claim 20, wherein the effective amount VEGF is about 72 μg/cm².

23. The method of claim 20, wherein the effective amount VEGF is about 216 μg/cm².

24. The method of claim 1, wherein the subject is human.

25. A method of accelerating wound healing in a human subject, the method comprising administering an effective amount of VEGF to a wound, wherein the administration of the effective amount of VEGF accelerates wound healing greater than 60% when compared a control and wherein the wound is present on the subject for about 4 weeks or more before administering the effective amount of VEGF.
